(19) United States
(12) Patent Application Publication
(10) Pub. No.: US 2007/0260258 A1
(43) Pub. Date: Nov. 8, 2007

(54) Access and delivery needle for percutaneous vertebroplasty

(76) Inventor: Robert Sommerich, Norton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/429,017

(22) Filed: May 5, 2006

(51) Int. Cl. A61F 2/00

(52) U.S. Cl. 606/92

(57) ABSTRACT

The vertebroplasty needle has two cannulae sharing a common distal end, wherein the cement injection cannula merges into a straight stylet cannula.

### BACKGROUND OF THE INVENTION

In vertebroplasty, the surgeon seeks to treat a compression fracture of a vertebral body by injecting bone cement such as PMMA into the fracture site. One clinical report describes mixing two PMMA precursor components (one powder and one liquid) in a dish to produce a viscous bone cement; filling 10 cc syringes with this cement, injecting it into smaller 1 cc syringes, and finally delivering the mixture into the desired area of the vertebral body through needles attached to the smaller syringes.

The conventional access needle used in a vertebroplasty procedure is a simple straight cannula extending along a single longitudinal axis, and has a handle formed near its proximal end. A single delivery port made from a luer connection is typically located at the proximal end of the cannula. The distal end of an injection syringe is typically attached to this delivery port via the luer connection. Typically, a stylet may also be placed within the cannula from the proximal end of the cannula in order to stabilize the cannula during insertion into the vertebral bone.

Because of the single axis nature of this conventional design, the injection syringe is typically connected to the proximal port of the cannula so that the injection syringe is also aligned along the same longitudinal axis. When this assembly is placed upon the prone patient's back, it typically sticks straight up in a vertical direction. Actuation of a vertically-disposed and vertically-displaced delivery syringe may be ergonomically challenging to the surgeon or clinician.

U.S. Pat. No. 6,916,292 (“Morawski”) discloses a bone biopsy needle having a single access port and a cannula that does not extend along a straight longitudinal axis, but rather curves near the proximal end. Because of its curved nature, this needle can not accommodate the insertion of a conventional straight stylet. The stylet is particularly important in percutaneous vertebroplasty procedures, as tracts of implant materials may be left in the body in unwanted locations if the access needle has not been cleared.

U.S. Pat. No. 6,302,852 (“Fleming III”) discloses a bone marrow biopsy needle including an outer cannula having a proximal end, a distal end, a hollow section therebetween and a handle portion associated with the proximal end, and an inner rod having a proximal end, a distal end and a handle cap. The handle portion further includes a grip enhancement member which is formed from a material distinct from at least a portion of the handle portion, such as rubber. The grip enhancement member may take the form of insert members which fit into cavities in the handle portion of the outer cannula. The grip enhancement member not only enhances the gripping surface of the bone marrow biopsy needle, but also provides cushioning for a user and adds weight to the handle portion to facilitate weight distribution throughout the outer cannula handle.

U.S. Pat. No. 6,875,183 (“Cervi”) discloses a biopsy needle for sampling bone marrow tissue, comprising a handle, and tissue sampling means comprising a sampling tube with a bore therein to receive a tissue sample, the outer surface of the sampling tube having an abrading formation extending in an axial direction along the tube, for abrading the sampled tissue, to permit the cannula tip to be laterally displaced whilst the tube is inserted into the bone marrow tissue. A biopsy needle for sampling bone marrow tissue, comprising a handle, tissue sampling means comprising a sampling tube with a bore therein to receive a tissue sample, and coupling means, separable from the needle, for coupling the needle to a rotary motor drive, whereby the needle is adaptable to both manual insertion and motor-assisted insertion.

U.S. Pat. No. 6,582,439 (“Sproul”) discloses an orthopedic surgical kit for inserting a biological material into the cancellous portion of bone by a minimally invasive technique has several components which are manually operated using a universal handle. The kit includes a docking needle used as a guide for placing a cannula in a bone. The cannula is filled with a biological material, for support or treatment of the bone, and the material is expressed from the cannula by a plunger.

### SUMMARY OF THE INVENTION

One object of the present invention is to provide the surgeon a more ergonomic means of delivering a viscous material through a needle.

The invention provides a more ergonomic approach to delivery of viscous materials used in percutaneous vertebroplasty and kyphoplasty procedures. The needle of the present invention includes a second off-angle cannula whose proximal end is used as the delivery port for an injection syringe. The distal end of this second needle then merges with the first cannula at an angle within the handle of the needle to provide fluid connection with the first cannula. This new needle provides a more horizontal posture to the injection syringe upon its connection with the off angle cannula and therefore provides a better ergonomic and tactile feel for implant delivery.

Therefore, in accordance with the present invention, there is provided an injection needle for use in percutaneous vertebroplasty, comprising:
a. a substantially longitudinal first tube having a throughbore, a proximal end portion defining a proximal end opening and a distal end portion defining a distal end opening,
b. a handle surrounding the proximal end portion of the first tube and having an outer surface, wherein the proximal end opening of the first tube opens onto the outer surface of the handle, and
   a second tube located at least partially within the handle and having a throughbore, a distal end in fluid connection with the first tube at a junction, and a proximal end defining a proximal end opening that opens onto the outer surface of the handle.

### DESCRIPTION OF THE FIGURES

FIG. 1A shows a cross-section of the needle of the present invention.

FIG. 1B, shows a cross-section of a needle, wherein the second tube is directed upwards from the first tube.

FIG. 1C shows a cross-section of needle of FIG. 1A with a stylet partially inserted therein.

FIG. 2 shows a cross-section of a needle of the present invention having a modular handle.

FIGS. 3A and 3B shows perspective views of the needle and syringe of the present invention in their respective assembled and exploded forms.

### DETAILED DESCRIPTION OF THE INVENTION

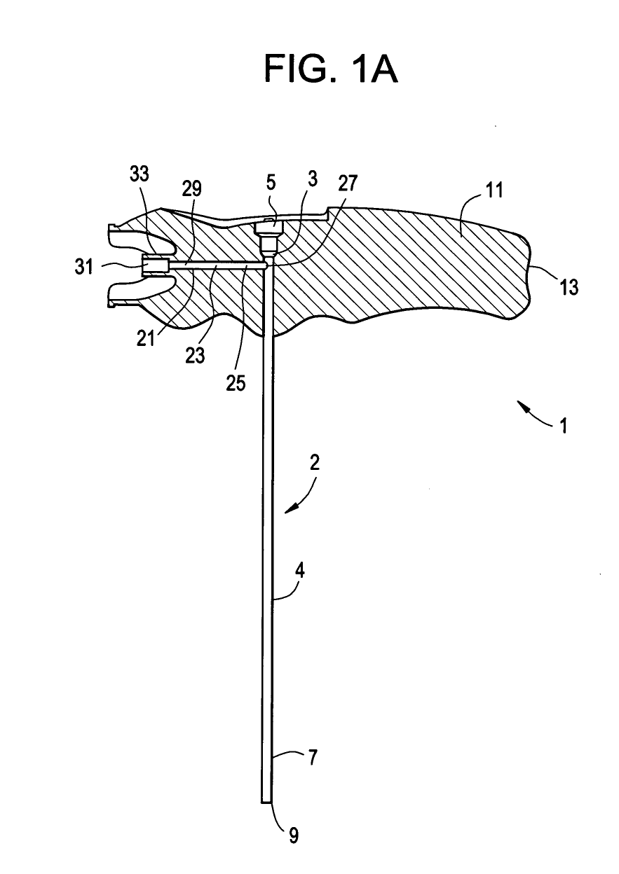

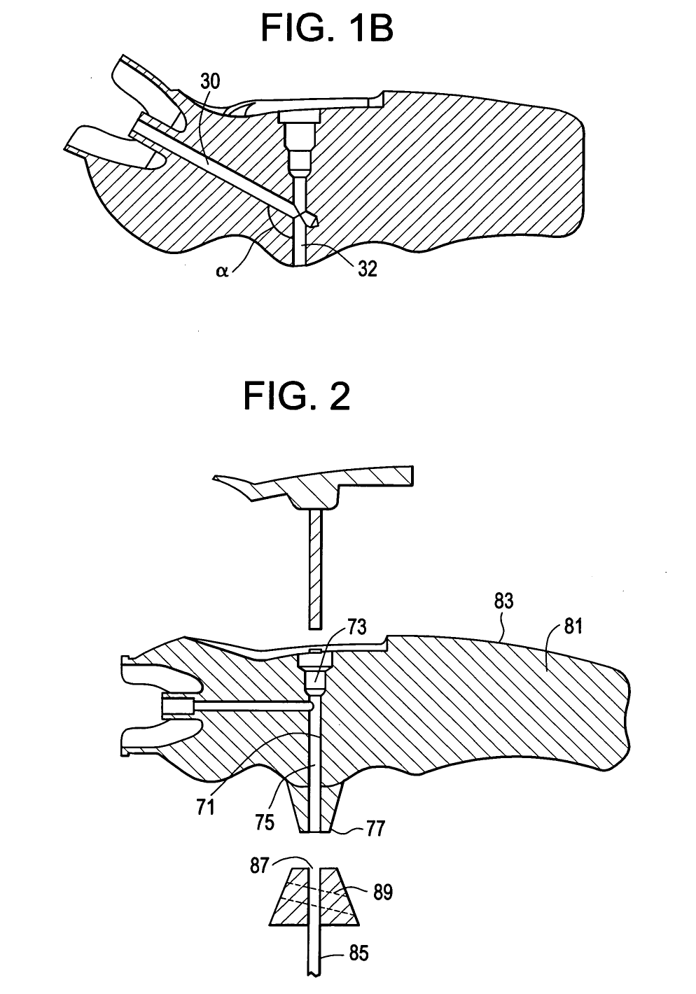

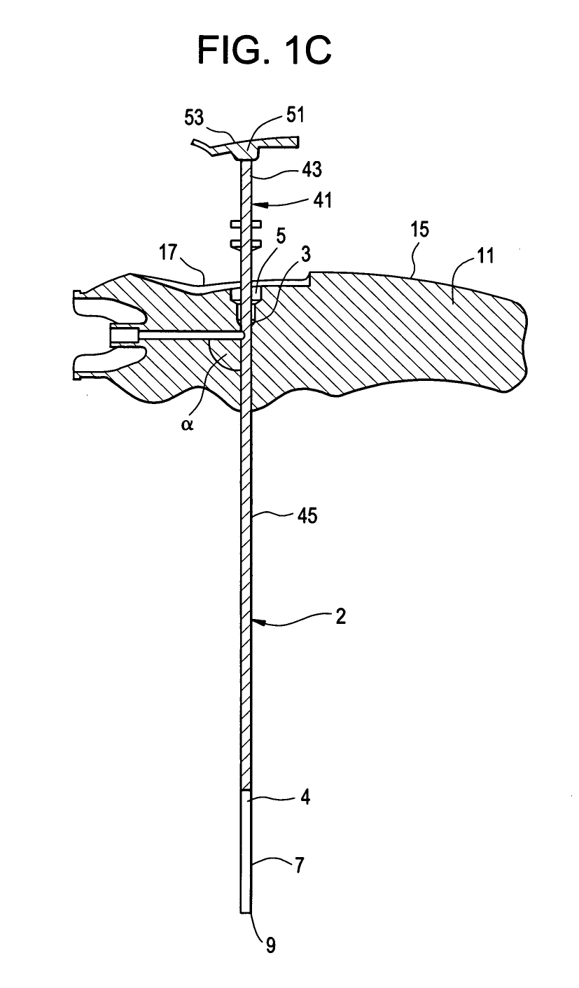

Now referring to FIGS. 1A, there is provided an injection needle 1 for use in percutaneous vertebroplasty, comprising:
a) a substantially longitudinal first tube 2 having a throughbore 4, a proximal end portion 3 defining a proximal end opening 5 and a distal end portion 7 defining a distal end opening 9,
b) a handle 11 surrounding the proximal end portion of the first tube and having an outer surface 13, wherein the proximal end opening of the first tube opens onto the outer surface of the handle, and
c) a second tube 21 located at least partially within the handle and having a throughbore 23, a distal end 25 in fluid connection with the first tube at a junction 27, and a proximal end 29 defining a proximal end opening 31 that opens onto the outer surface of the handle.

In some embodiments, the tube component of the present invention can be formed of a material that is distinct from the surrounding handle. In other embodiments, the tube is formed by simply making a throughbore in the surrounding handle.

When assembled, the needle comprises a bone access port at its distal end, and two ports on its proximal end. The proximal end of the needle includes a first pathway to the bone access port to accommodate a stylet and a second pathway to the bone access port for attachment of a cement injection system. The second pathway is more horizontally disposed than the first pathway, allowing for improved positioning and ergonomics during delivery of the viscous implant material. The first and second tubes meet at a junction and form an angle α. FIG. 1A shows a substantially horizontal attachment of the delivery system to the handle (α=90°); however, a more ergonomic approach may have the second pathway directed at an angle (i.e., angled up (α>90°) or down (α<90°)) from the horizontal.

Now referring to FIG. 1B, there is provided an embodiment of the present invention wherein the second tube 30 is directed upwards from the first tube 32 whereby angle α is greater than 90 degrees.

In other embodiments, the second pathway is directed at a lateral angle (i.e., to the left or right of the handle). In other embodiments, the angle is rotatably adjustable.

This invention provides multiple pathways to the bone access port of an access needle for PV procedures, one of which is an access pathway for a straight stylet. It advantageously provides the surgeon with better positioning in relation to the proximal injection port during delivery of the implant material, while at the same time including the required stylet access for PV procedures.

The proper orientation of the proximal access port and delivery system may also eliminate the need for the extension tubing. Since the angled design of the present invention displaces the surgeon's hands laterally (in relation to the longitudinal axis of the second tube), it may also allow the surgeon's hands to be moved out of the fluoroscopy field during implant delivery without the use of an extension tube. Because extension tubes often impose additional pressure requirements upon the injection device, the elimination of the extension tube may reduce the pressure requirements to deliver the viscous implant material.

In some embodiments, a one-way valve or silicone plug is provided to prohibit backflow of implant material through the second proximal pathway. Typically, the one-way valve is located in the first tube at a location proximal of the junction. Alternatives to a one-way valve or silicone plug include a thumb-trigger that opens the second pathway when depressed, or a ball-plunger that retracts when the stylet is placed within the second pathway.

In order to prevent injection of material while the stylet is in place, the handle includes a feature to fully or partially cover the luer connection 33 (shown open in FIG. 1A) associated with the injection port. An ergonomic handle component is provided that is able to separate into a delivery section and a stylet to partially or fully cover the luer connector to the delivery device so that implant material may not be injected while the stylet is in place. Thereafter, when the stylet is removed, there is full exposure of the luer connection.

In order to improve ergonomics, the luer connector to the delivery system may be placed at an angle to the handle or adapted to allow rotation to a preferred position. The use of allowable rotation provides a single beveled needle to be optimally oriented (typically anteriorly) while allowing the proximal inlet port to be oriented towards the surgeon. This not only provides improved ergonomics, it also may allow for elimination of extension tubing from the system.

Therefore, in accordance with the present invention, there is provided (claim 12) an injection needle for use in percutaneous vertebroplasty, comprising:
a. a substantially longitudinal first tube having a throughbore, a proximal end portion defining a proximal end opening and a distal end portion defining a distal end opening,
b. a handle surrounding the proximal end portion of the first tube and having an outer surface, wherein the proximal end opening of the first tube opens onto the outer surface of the handle, and
c. a rotatable luer connection disposed at the distal end opening of the first tube

In order to increase surgeon visibility to the bone access site, the stylet may be removed from the handle. In so doing, most of the bulk of the handle is likewise removed.

Also, the handle may include two or more components that contain the stylet and the delivery access tube to the vertebral body. The handle may have a luer connection in the injection system that is partially or full contained within the removal stylet component.

Therefore, and now referring to FIG. 1C, in accordance with the present invention, there is provided an injection needle for use in percutaneous vertebroplasty, comprising:
a) a substantially longitudinal outer tube 2 having a throughbore 4, a proximal end portion 3 defining a proximal end opening 5 and a distal end portion 7 defining a distal end opening 9,
b) an outer handle 11 surrounding the proximal end portion of the outer tube and having a proximal surface 15 having a recess 17, wherein the proximal end opening of the outer tube opens onto the proximal surface of the handle within the recess,
c) a substantially longitudinal inner stylet 41 having a proximal end portion 43 and a distal end portion 45, and
d) an inner handle 51 surrounding the proximal end portion of the inner stylet,
   wherein the inner stylet is received within the throughbore of the outer tube, and wherein the inner handle fits substantially within the recess of the outer handle.

Preferably, the inner handle has a proximal surface 53, the proximal surface of the the outer handle forms a first plane, and the proximal surface of the inner handle lies substantially the first plane when the inner stylet is received within the throughbore of the outer tube

During the delivery of the cement and subsequent fluoroscopic assessment thereof, it appears that the large dimensions of the handle may sometimes obstruct the clinician's view of the injection site. This obstruction may be particularly problematic when the surgeon seeks to use the stylet as a tamp for tamping the bone cement that remains in the delivery tube. Therefore, in some embodiments of the present invention, the handle is manufactured as a modular component of the injection needle. The handle can be modularized by providing a first luer attachment at the distal end of the substantially longitudinal first tube, and a second luer attachment adapted to mate with the first luer attachment upon the delivery tube. Now referring to FIG. 2, in accordance with the present invention, there is provided (claim 28) an injection needle for use in percutaneous vertebroplasty, comprising:
a) a substantially longitudinal first tube 71 having a throughbore, a proximal end portion 73 defining a proximal end opening and a distal end portion 75 defining a distal end opening having a first luer connection thereon 77, and
b) a handle 81 surrounding the proximal end portion of the first tube and having an outer surface 83, wherein the proximal end opening of the first tube opens onto the outer surface of the handle, and
c) a delivery tube 85 having a throughbore and a proximal end portion 87 defining a proximal end opening having a second luer connection 89 thereon that mates with the first luer connection.
   In use, the surgeon can inject cement through the needle, and then remove the handle in order to have better visualization of the operative site when using the stylet. Alternatively, the surgeon can inject the cement through the delivery tube 85 and second luer 89 without the handle attached.

During the manufacturing of long tubes or needles used in PV, there may be manufacturing remnant materials inherently remaining within the tube or on the stylet. This is typically seen as grey material within the cement during injection. While cleaning with brushes or high pressure fluids assists in removing some of the remnant material, there is typically some remnant material left behind. The present invention seeks to reduce the delivery to the patient of debris remaining from the manufacturing process. This is accomplished by providing a coating on the inside surface of the needle to encapsulate or cover the inside of the tube (or outside surface of the stylet). The inner surface of the needle may be coated with a variety of materials (i.e. PTFE (Teflon), or its surface may be conditioned by a variety of manufacturing methods (such as electropolishing or plating) that may cover up undesirable manufacturing debris present upon the inside of the needle, thereby precluding their delivery to the patient. In addition, such a coating may reduce the friction on the inner surface of the cannula, thereby easing the flow of the cement passing through the needle and requiring less pressure to deliver the implant material.

Therefore, in accordance with the present invention, there is provided (claim 13) injection needle for use in percutaneous vertebroplasty, comprising:
a) a substantially longitudinal first tube having an inner surface defining a throughbore, a proximal end portion defining a proximal end opening and a distal end portion defining a distal end opening,
b) a handle surrounding the proximal end portion of the first tube and having an outer surface, wherein the proximal end opening of the first tube opens onto the outer surface of the handle, and
   wherein the inner surface of the first tube is coated, electropolished or electroplated.

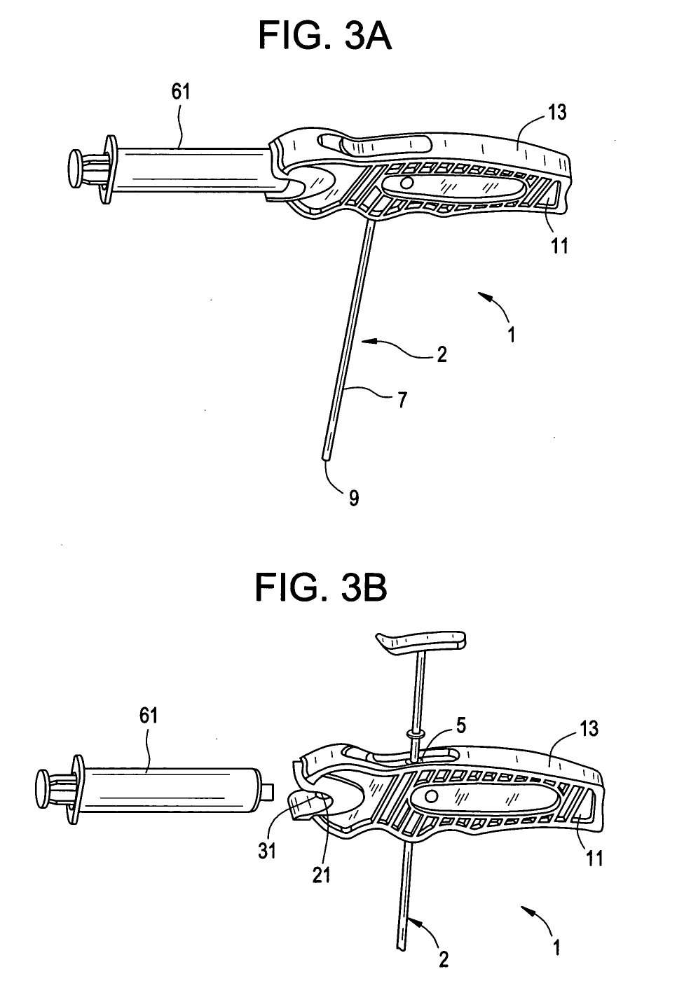

Now referring to FIGS. 3A-3B, in use, the device of the present invention is connected to a syringe contain a bone paste, and the paste is injected into the bone via the needle. The bone paste used in the vertebroplasty procedure may be any material typically used to augment vertebral bodies.

Therefore, in accordance with the present invention, there is provided (claim 20) a device for use in percutaneous vertebroplasty, comprising:
a) an injection needle 1 comprising:
   i) a substantially longitudinal first tube 2 having a throughbore, a proximal end portion defining a proximal end opening 5 and a distal end portion 7 defining a distal end opening 9,
   ii) a handle 11 surrounding the proximal end portion of the first tube and having an outer surface 13, wherein the proximal end opening of the first tube opens onto the outer surface of the handle, and
   ii) a second tube 21 located at least partially within the handle and having a throughbore, a distal end in fluid connection with the first tube at a junction, and a proximal end defining a proximal end opening 31, and
b) a syringe 61 fluidly connected to the proximal end opening of the second tube.

In some embodiments, the syringe can be replaced with a delivery device having a material reservoir and tubing adapted to deliver the cement from the reservoir to the proximal end opening of the second tube.

Preferred bone pastes include bone cements (such as acrylic-based bone cements, such as PMMA-based bone cements), pastes comprising bone particles (either mineralized or demineralized or both; and either autologous, allogenic or both), and ceramic-based bone cements (such as HA and TCP-based pastes).

I claim:

1. An injection needle for use in percutaneous vertebroplasty, comprising:
a) a substantially longitudinal first tube having a throughbore, a proximal end portion defining a proximal end opening and a distal end portion defining a distal end opening,
b) a handle surrounding the proximal end portion of the first tube and having an outer surface, wherein the proximal end opening of the first tube opens onto the outer surface of the handle, and
c) a second tube located at least partially within the handle and having a throughbore, a distal end in fluid connection with the first tube at a junction, and a proximal end defining a proximal end opening that opens onto the outer surface of the handle.

2. The needle of claim 1 wherein the second tube is disposed at an angle α to the substantially longitudinal first tube.

3. The needle of claim 2 wherein angle α is substantially 90°.

4. The needle of claim 2 wherein angle α is greater than 90°.

5. The needle of claim 2 wherein angle α is less than 90°.

6. The needle of claim 2 wherein the second tube has a luer connection upon its proximal end opening.

7. The needle of claim 6 wherein the luer connection is oriented at an angle to the second tube.

8. The needle of claim 1 further including:
d. a stylet sized to fit within the throughbore of the first tube.

9. The needle of claim 1 wherein the needle further comprises a backflow prevention means located in the first tube at a location proximal of the junction.

10. The needle of claim 9 wherein the backflow prevention means is selected from the group consisting of a one-way valve, a silicone plug, a thumb trigger and a ball-plunger.

11. The needle of claim 1 further comprising a coating upon the throughbore of the first tube.

12. An injection needle for use in percutaneous vertebroplasty, comprising:
a. a substantially longitudinal first tube having a throughbore, a proximal end portion defining a proximal end opening and a distal end portion defining a distal end opening,
b. a handle surrounding the proximal end portion of the first tube and having an outer surface, wherein the proximal end opening of the first tube opens onto the outer surface of the handle, and
c. a rotatable luer connection disposed at the distal end opening of the second tube.

13. An injection needle for use in percutaneous vertebroplasty, comprising:
a. a substantially longitudinal first tube having an inner surface defining a throughbore, a proximal end portion defining a proximal end opening and a distal end portion defining a distal end opening,
b. a handle surrounding the proximal end portion of the first tube and having an outer surface, wherein the proximal end opening of the first tube opens onto the outer surface of the handle, and
wherein the inner surface of the first tube is coated, electropolished or electroplated.

14. The needle of claim 8 wherein the inner surface of the first tube is coated with a coating.

15. The needle of claim 9 wherein the coating is PTFE.

16. The needle of claim 8 wherein the inner surface of the first tube is electropolished.

17. The needle of claim 8 wherein the inner surface of the first tube is electroplated.

18. A device for use in percutaneous vertebroplasty, comprising:
a) an injection needle comprising:
i) a substantially longitudinal first tube having a throughbore, a proximal end portion defining a proximal end opening and a distal end portion defining a distal end opening,
ii) a handle surrounding the proximal end portion of the first tube and having an outer surface, wherein the proximal end opening of the first tube opens onto the outer surface of the handle, and
iii) a second tube located at least partially within the handle and having a throughbore, a distal end in fluid connection with the first tube at a junction, and a proximal end defining a proximal end opening, and
b) a cement delivery device fluidly connected to the proximal end opening of the second tube.

19. The device of claim 18 wherein the syringe contains a bone paste material used to augment a vertebral body.

20. The device of claim 19 wherein the material is a bone cement.

21. The device of claim 19 wherein the material is a PMMA bone cement.

22. The device of claim 19 wherein the material comprises bone particles.

23. The device of claim 19 wherein the material is a ceramic-based bone cement.

24. The device of claim 18 wherein the injection needle further comprises:
iv) a handle surrounding the proximal end portion of the first tube and having an outer surface, wherein the proximal end opening of the first tube opens onto the outer surface of the handle.

25. The device of claim 24 wherein the proximal end opening of the second tube opens onto the outer surface of the handle.

26. An injection needle for use in percutaneous vertebroplasty, comprising:
a) a substantially longitudinal first tube having a throughbore, a proximal end portion defining a proximal end opening and a distal end portion defining a distal end opening having a first luer connection thereon, and
b) a handle surrounding the proximal end portion of the first tube and having an outer surface, wherein the proximal end opening of the first tube opens onto the outer surface of the handle, and
c) a delivery tube having a throughbore and a proximal end opening having a second luer connection thereon that mates with the first luer connection.
